(19) United States
(12) Patent Application Publication
(10) Pub. No.: US 2014/0378936 A1
(43) Pub. Date: Dec. 25, 2014

(54) UNDERGARMENT WITH REMOVABLE LINER

(71) Applicant: Tailored Technologies, Inc., Winston-Salem, NC (US)

(72) Inventor: Fredrica V. Coates, Winston-Salem, NC (US)

(21) Appl. No.: 14/310,434

(22) Filed: Jun. 20, 2014

Related U.S. Application Data
(60) Provisional application No. 61/837,698, filed on Jun. 21, 2013.

(51) Int. Cl. A61F 13/72; A61F 13/66

(52) U.S. Cl. CPC A61F 13/72; A61F 13/665; A61F 2013/15121
USPC 604/396

(57) ABSTRACT

An undergarment including a pocket extending from an anterior crotch portion of the undergarment to a posterior crotch portion of the undergarment, the pocket closed along three sides and opening in a direction of the anterior crotch, a hood arranged on the anterior crotch portion concealing the opening of the pocket, and an absorbent pad removable from the pocket, wherein the hood is configured to be pulled aside to expose the opening of the pocket to remove and insert the absorbent pad from the pocket.

### CROSS-REFERENCE TO RELATED APPLICATION

This application claims priority from U.S. Application No. 61/837,698 filed Jun. 21, 2013, the entirety of which is incorporated by reference herein.

### TECHNICAL FIELD AND BACKGROUND OF THE INVENTION

The present invention relates generally to the field of undergarments (i.e., underwear including panties, briefs, boxers, boxer briefs, etc.), and more particularly, to comfortable underwear having a removable liner configured to absorb, retain and prevent bodily fluids from reaching and/or penetrating though undergarment.

Urinary incontinence is the involuntary leakage of urine, which often affects many middle-aged adults and geriatrics. Continence and micturition involve a balance between urethral closure and detrusor muscle activity, and when this balance is lost (e.g., a weakening of the urethra), urinary incontinence results.

Urinary incontinence is a distressing problem that may have a profound impact on quality of life. For example, numerous types of urinary incontinence exist including, but not limited to, stress incontinence, urge incontinence, overflow incontinence, functional incontinence, giggle incontinence, and post-void dribbling incontinence. In women, urinary incontinence often occurs after childbirth due to loss of urethra support as a consequence of damage to pelvic support structures during childbirth. Likewise, in geriatrics, urinary incontinence often occurs due to weakening of the urethra due to aging.

In the past, those afflicted with urinary incontinence relied on either complicated elective surgeries to potentially correct this distressing problem or specialized undergarments to hide the symptoms associated with urinary incontinence. For example, those afflicted with urinary incontinence often use adult diapers to hide the symptoms of this malady. However, many of these adult diapers are large, bulky, and uncomfortable for a wearer, and many of these adult diapers are very difficult to conceal underneath clothing.

### BRIEF SUMMARY OF THE INVENTION

Therefore, a need exists for an undergarment (e.g., underwear or panty) configured to hide the symptoms of urinary incontinence while simultaneously being comfortable to wear and easy to conceal underneath clothing.

In a particular embodiment, the undergarment includes a pocket extending from a wearer's anterior crotch to a wearer's posterior crotch, a hood positioned against the wearer's anterior crotch covering the front opening of the pocket, and an absorbent pad removable from the pocket. In certain aspects, the hood is configured to be pulled aside (i.e., raised, moved, displaced, stretched, etc.) to provide access to the underlying opening of the pocket to remove the absorbent pad from the pocket and to insert the absorbent pad into the pocket. In certain aspects, the pocket is closed on three sides and is open adjacent the wearer's anterior crotch (i.e, front of the garment).

In certain aspects, the hood covers a first portion of the absorbent pad and the pocket covers a second portion of the absorbent pad.

In certain aspects, the hood is wider than the opening of the pocket.

In certain aspects, the hood has an arcuate shape.

In certain aspects, the pocket and hood are sewn into the undergarment using, for example, lock stitches.

In certain aspects, the absorbent pad includes a fluid-absorbent material configured to absorb bodily fluids and a waterproof backing configured to trap bodily fluids within the absorbent pad such that bodily fluid leakage from the undergarment is eliminated or minimized.

In certain aspects, the absorbent pad is configured to be wider adjacent the wearer's anterior crotch and tapers in width in the direction of the wearer's posterior crotch, thus providing greater surface area for absorption/protection in the anterior crotch area.

In certain aspects, the absorbent pad may taper in thickness in the direction of the posterior crotch.

Also disclosed herein is an undergarment having an absorbent pad permanently incorporated within the undergarment such that the absorbent pad extends from a wearer's anterior crotch to a wearer's posterior crotch and the absorbent pad is configured to be thicker in the wearer's anterior crotch and tapers in thickness as the absorbent pad extends towards the wearer's posterior crotch.

In certain aspects, the absorbent pad tapers in width as the absorbent pad extends from a wearer's anterior crotch to a wearer's posterior crotch.

In certain aspects, the absorbent pad includes a fluid-absorbent material configured to absorb bodily fluids and a waterproof backing configured to trap bodily fluids within the absorbent pad such that bodily fluid leakage from the undergarment is eliminated or minimized.

Embodiments of the invention can include one or more or any combination of the above features, aspects and configurations.

Additional features, aspects and advantages of the invention will be set forth in the detailed description which follows, and in part will be readily apparent to those skilled in the art from that description or recognized by practicing the invention as described herein. It is to be understood that both the foregoing general description and the following detailed description present various embodiments of the invention, and are intended to provide an overview or framework for understanding the nature and character of the invention as it is claimed. The accompanying drawings are included to provide a further understanding of the invention, and are incorporated in and constitute a part of this specification.

### BRIEF DESCRIPTION OF THE DRAWINGS

Features, aspects and advantages of the present invention are understood when the following description of the invention is read with reference to the accompanying drawings, in which:

FIG. 1 is a front view showing the exterior of an exemplary undergarment according to an embodiment of the invention;

FIG. 2 is a detailed view of the inside of the exemplary underwear showing the absorbent liner removed from the pocket;

FIG. 3 is a detailed view of the liner pocket showing the liner partially inserted into the pocket;

FIG. 4 is a detailed view of the liner pocket showing the liner partially inserted into the pocket;

FIG. 5 is a detailed view of the liner pocket showing the liner completely inserted into the pocket; and

FIG. 6 is a second embodiment showing the liner permanently sewn into the underwear.

### DETAILED DESCRIPTION OF THE INVENTION

The present invention will now be described more fully hereinafter with reference to the accompanying drawings in which exemplary embodiments of the invention are shown. However, the invention may be embodied in many different forms and should not be construed as limited to the representative embodiments set forth herein. The exemplary embodiments are provided so that this disclosure will be both thorough and complete, and will fully convey the scope of the invention and enable one of ordinary skill in the art to make, use and practice the invention. Like reference numbers refer to like elements throughout the various drawings.

As used herein, the term “anterior crotch” refers to the area immediately forward of and/or above the wearer's genitalia (e.g., the pubic region immediately above the male or female genitalia). As used herein, the term “posterior crotch” refers to the wearer's perineum.

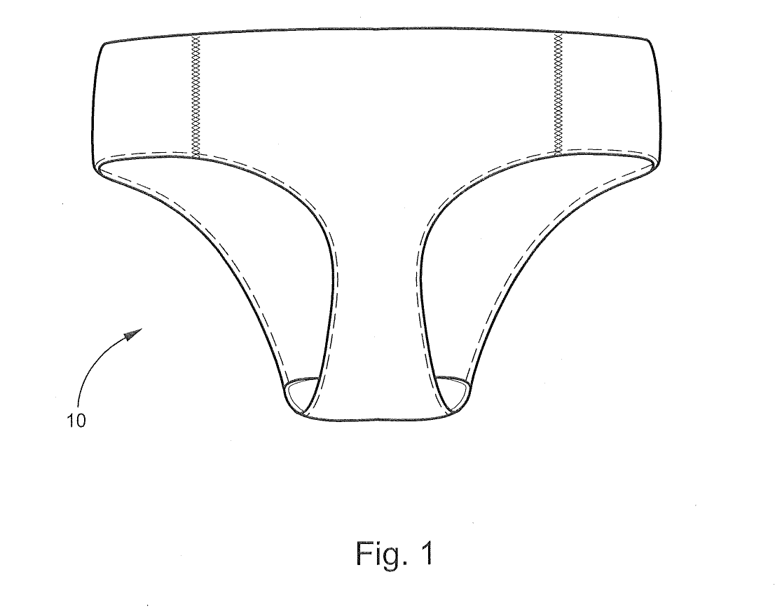

Disclosed is an undergarment that is capable of hiding the symptoms of urinary incontinence while simultaneously being comfortable to wear and easy to conceal beneath clothing. Referring to FIG. 1, a first embodiment of an undergarment (e.g,.underwear) suitable for being fitted with a liner pocket for receiving a removable liner therein is shown generally at reference numeral 10. The undergarment 10 (e.g., underwear) may have other forms including, but not limited to, briefs, boxer briefs, thongs, etc. The undergarment may be made from cotton, spandex, lycra, rayon, polyester, or any combination thereof, and is preferably made from any material comfortable to a wearer. The undergarment may further include, for example, an elastic waistband, elastic leg bands, or a combination thereof As further shown in FIG. 1, the pocket and liner are substantially concealed when viewed from the front.

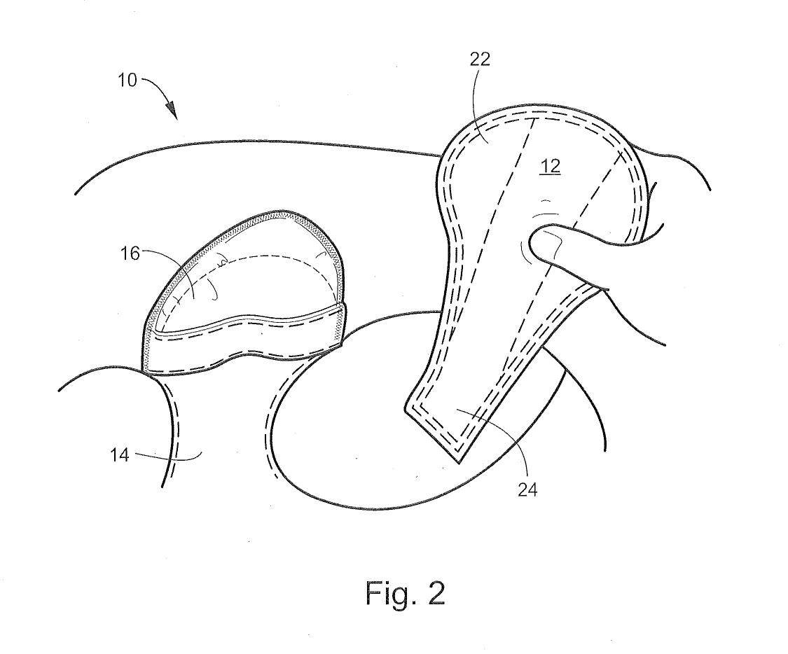

As illustrated in FIGS. 2-5, the undergarment 10 includes a pocket 14 extending from about a wearer's anterior crotch to about a wearer's posterior crotch, and a hood 16 arranged adjacent the wearer's anterior crotch. In certain aspects, the hood 16 covers the opening 18 of the pocket, and in certain aspects, the pocket 14 and hood 16 are configured to receive and cooperatively retain a removable liner 12, also referred to herein as the “absorbent pad.” For example, as shown in FIG. 2, the liner 12 is a reusable absorbent pad removable from the pocket 14 of the crotch portion of the underwear 10. The liner 12 has a shape wider at the front 22 (i.e., the portion configured to be adjacent to a wearer's anterior crotch) and narrower at the back 24 (i.e., the portion configured to be adjacent to a wearer's posterior crotch) to capture the spread of fluid where most needed. In other words, the liner 12 tapers in width in the direction of the posterior crotch. In certain aspects, the liner 12 may also be thicker in the front or at the top for greater fluid absorption in the anterior crotch. Thus, the liner 12 may taper in thickness in the direction of the posterior crotch.

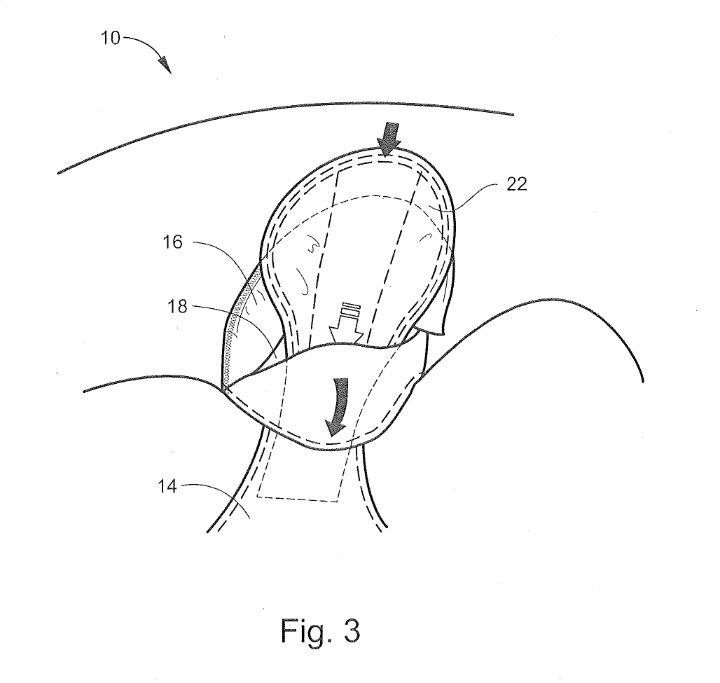

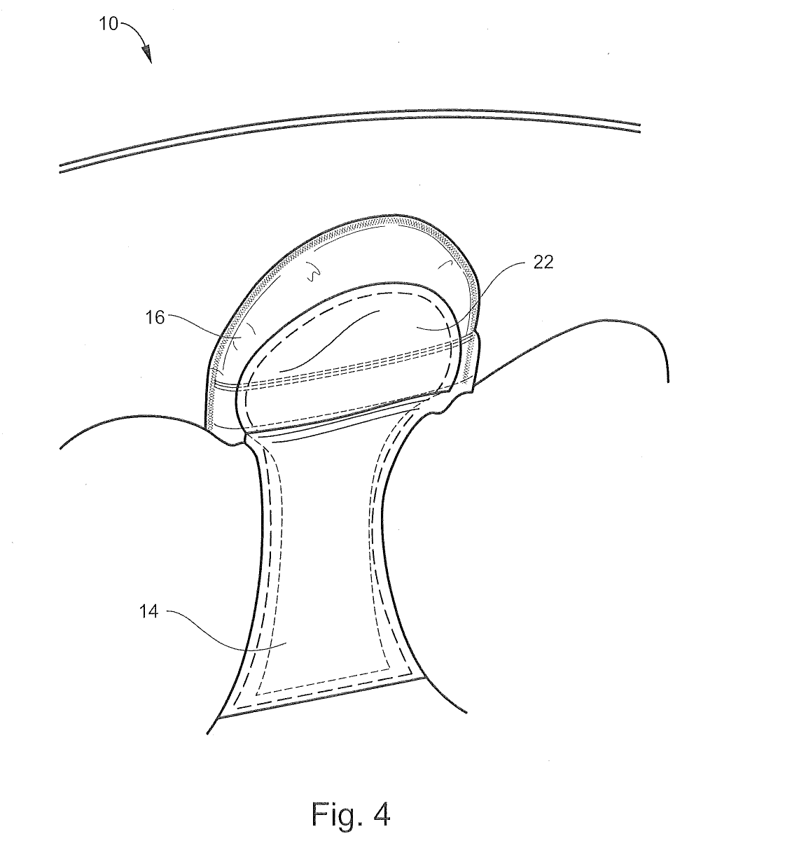

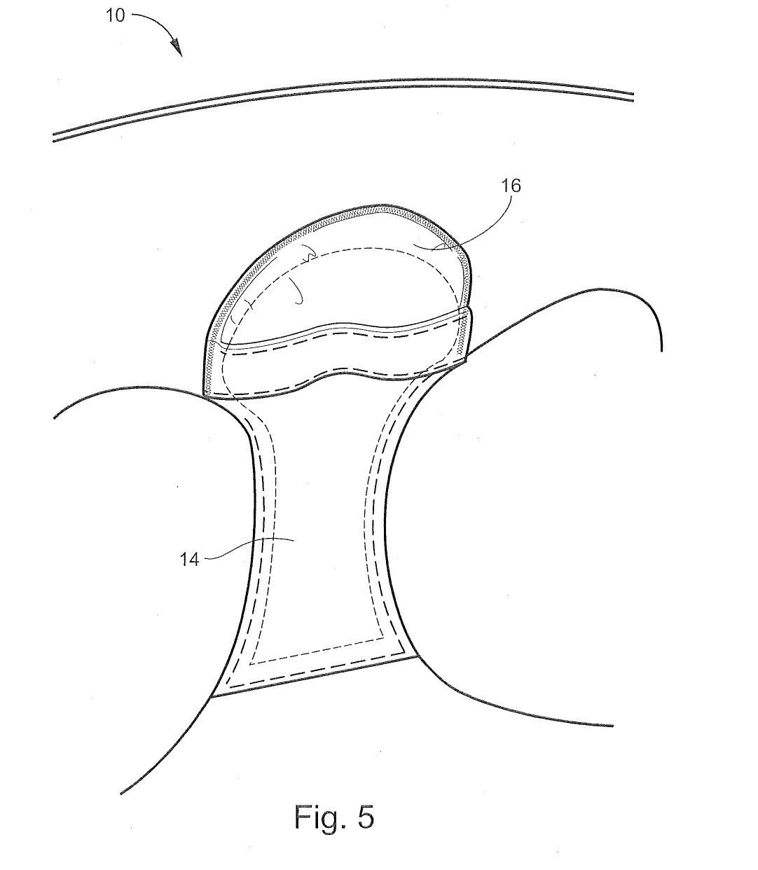

Referring specifically to FIGS. 2 and 3, the pocket 14 and hood may substantially overlap, thus generally providing “three-layers” of protection. Referring specifically to FIGS. 4 and 5, the pocket may terminate in horizontal edge positioned just beneath the hood 16, and thus slightly overlapping.

The liner 12 is preferably washable, and preferably includes a fluid-absorbent material and a waterproof backing. The fluid-absorbent material may include a woven or non-woven fabric that includes cotton, a water-absorbent polymeric material, or a combination thereof. The waterproof backing may include a waterproof plastic made from, for example, polyethylene, polystyrene, polyvinylchloride, or a combination thereof In the alternative, the waterproof backing may include either a woven or non-woven fabric treated with a water-repellant (e.g., a silicone containing water repellant or a fluorochemical water repellant such as polytetrafluoroethylene). When inserted in the undergarment, the liner 12 preferably minimizes or eliminates fluid leakage from the undergarment such that bodily fluids do not penetrate through the undergarment and/or through a wearer's clothes.

The absorbent side is preferably inserted facing the wearer, with the waterproof backing facing away from the wearer. The pocket 14 is elongate and substantially covers the crotch of the underwear and preferably extends from a wearer's anterior crotch to a wearer's posterior crotch such that the pocket begins preferably above the wearer's genitals and ends at the wearer's perineum. The pocket 14 and hood 16 together are generally shaped to correspond to the shape of the liner 12, and the shape of any component may be customized to fit within the shape, style and bounds of the undergarment.

In a preferred embodiment, the pocket 14 is sewn to the undergarment along three sides and open along one side, preferably the front concealed from view beneath the hood 16. The hood 16 covers a portion of the pocket 14 and the opening thereto and can be pulled apart from, stretched, lifted, raised, etc. to reveal the underlying opening and provide access thereto to insert and remove the liner 12. When the hood is “open,” the opening to the pocket is exposed. When the hood is “closed,” the opening is concealed.

Referring to FIGS. 3-5, various stages of insertion of the liner 12 into the pocket 14 are shown. FIG. 3 shows the liner 12 being inserted into the pocket 14. For example, as shown in FIG. 3, the hood 16 may be configured to be pulled aside to provide access to the opening of the pocket 18. The liner 12 is inserted into the pocket 14 and pushed downwardly towards the portion of the undergarment configured to be adjacent to a wearer's posterior crotch. Specifically, FIG. 3 shows the narrow portion of the liner 24 (i.e., the portion configured to be closest to a wearer's posterior crotch) partially inserted into the pocket 14. However, in FIG. 3, the portion of the liner 22 configured to be adjacent to a wearer's anterior crotch remains exposed and is not covered by the hood. FIG. 4 shows the entire narrow portion of the liner 24 inserted into the pocket 14 and the wide portion of the liner 22 (i.e., the portion configured to be adjacent to the wearer's anterior crotch) remaining outside of the hood 16. FIG. 5 shows the liner 12 fully inserted into the pocket 14 and completely covered by the hood 16 and the pocket 14. In this aspect, the hood 16 covers a first portion of the liner (e.g., the wide portion of the liner 22) and the pocket 14 covers a second portion of the liner (e.g., the narrow portion of the liner 24).

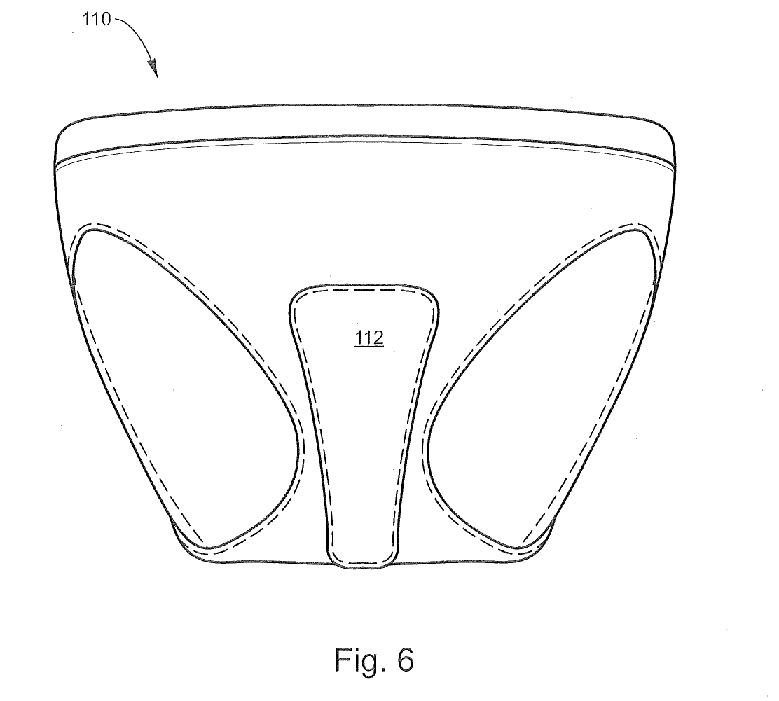

FIG. 6 shows an alternative embodiment of an undergarment 110 including an absorbent pad 112 permanently incorporated within the undergarment such that the absorbent pad extends from a wearer's anterior crotch to a wearer's posterior crotch and the absorbent pad is configured to be wider and/or thicker in the wearer's anterior crotch and tapers in width and/or thickness as the absorbent pad extends towards the wearer's posterior crotch. For example, in certain aspects, the fabric sewn (e.g., via lock stitching) in the center of the underwear is 100% cotton laminated and then sewn to the crotch of the underwear. In this embodiment, the fabric provides additional padding and absorbency, but is not removable from the underwear. In this embodiment, the absorbent pad preferably tapers in width as the absorbent pad extends from a wearer's anterior crotch to a wearer's posterior crotch. In this aspect, the absorbent pad preferably traps bodily fluids within the absorbent pad such that bodily fluid leakage from the undergarment is minimized.

The foregoing description provides embodiments of the invention by way of example only. It is envisioned that other embodiments may perform similar functions and/or achieve similar results. Any and all such equivalent embodiments and examples are within the scope of the present invention.

What is claimed is:

1. An undergarment, comprising:
a pocket extending from an anterior crotch portion of the undergarment to a posterior crotch portion of the undergarment, the pocket closed along three sides and opening in a direction of the anterior crotch;
a hood arranged on the anterior crotch portion concealing the opening of the pocket; and
an absorbent pad removable from the pocket;
wherein the hood is configured to be pulled aside to expose the opening of the pocket to remove and insert the absorbent pad from the pocket.

2. The undergarment of claim 1, wherein the hood covers a first portion of the absorbent pad and the pocket covers a second portion of the absorbent pad when the absorbent pad is fully inserted into the undergarment.

3. The undergarment of claim 1, wherein the hood is wider than the opening of the pocket.

4. The undergarment of claim 1, wherein the hood has an arcuate shape.

5. The undergarment of claim 1, wherein the pocket and the hood are sewn into the undergarment.

6. The undergarment of claim 1, wherein the absorbent pad includes a fluid-absorbent material and a waterproof backing.

7. The undergarment of claim 1, wherein the absorbent pad is wider at the anterior crotch portion and tapers in width in the direction of the posterior crotch portion.

8. The undergarment of claim 1, wherein the absorbent pad is thicker at the anterior crotch portion and tapers in thickness in the direction of the posterior crotch portion.

9. The undergarment of claim 1, wherein the hood and the pocket together define a shape that corresponds to a shape of the removable liner, and wherein the removable liner is wider in the area of the anterior crotch than in the area of the posterior crotch of the undergarment.

10. The undergarment of claim 1, wherein the absorbent pad is a multi-layered knitted fabric wherein the layers are stitched together.

11. An undergarment comprising an absorbent pad sewn on the undergarment and extending from an anterior crotch portion of the undergarment to a posterior crotch portion of the undergarment, the absorbent pad wider near the anterior crotch portion and tapering in width in a direction of the posterior crotch portion, the absorbent pad configured to absorb and retain bodily fluid.

12. The undergarment of claim 11, wherein the absorbent pad tapers in thickness in the direction of the posterior crotch portion.

13. The undergarment of claim 11, wherein the absorbent pad comprises a fluid-absorbent material and a waterproof backing, the fluid-absorbent material facing a wearer and the waterproof backing facing the undergarment.
